(19) United States
(12) Patent Application Publication
(10) Pub. No.: US 2004/0229914 A1
(43) Pub. Date: Nov. 18, 2004

(54) Novel crystalline form-VI of donepezil hydrochloride and process for the preparation thereof

(75) Inventors: Manne Satyanarayana Reddy, Erragadda (IN); Sajja Eswaraiah, Hyderabad (IN); Mathad Vijayavitthal Thippannachar, Hyderabad (IN); Elati Ravirama Chandrashekar, Hyderbad (IN); Podichetty Anil Kumar, Risala Bazar Secunderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/816,798

(22) Filed: Apr. 2, 2004

(30) Foreign Application Priority Data
Apr. 2, 2003 (IN) 279/MAS/2003

(51) Int. Cl. C07D 211/06; A61K 31/445

(52) U.S. Cl. 514/319; 546/206

(57) ABSTRACT

The invention relates to novel crystalline form VI of Donepezil hydrochloride and process for preparing the crystalline form VI, which is a simple, non-hazardous and commercially viable process.

### FIELD OF THE INVENTION

The present invention relates to novel crystalline form-VI of Donepezil hydrochloride. Donepezil hydrochloride is chemically known as 1-benzyl-4-[(5,6-dimethoxy-1-indanon)-2-yl]methyl-piperidine hydrochloride, which can be depicted by Formula-I.

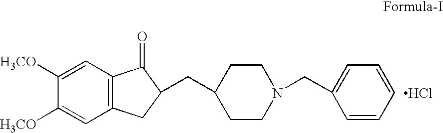

The present invention also relates to the process for the preparation of Novel crystalline form-VI of Donepezil hydrochloride.

Donepezil hydrochloride is having an excellent action as a medicament, disclosed in U.S Pat. No. 4,895,841 or EP-A 296,560, specifically as a prophylactic and therapeutic agent for senile dementia, and in particular as a prophylactic and therapeutic agent for Alzheimer's disease. And the said patent also discloses an industrial process for producing Donepezil hydrochloride.

### BACKGROUND OF THE INVENTION

Donepezil hydrochloride is a selective inhibitor of acetyl cholinesterase and is the first promising agent with this mode of action for the treatment of mild to moderate dementia of Alzheimer's type. This drug was approved first in US in 1997 and later in 7 countries and has provided an effective remedy for this central nervous system illness.

U.S. Pat. No. 4,895,841 discloses in experimental section (Example-4) that recrystallization of the crude Donepezil hydrochloride from a mixture of methanol and isopropyl ether afforded a purified Donepezil hydrochloride having melting range between 211 and 212° C.

Further, there are two granted patents, viz. U.S. Pat. No. 6,140,321 and U.S. Pat. No. 5,985,864 and two international applications under PCT, viz. WO 97/46526 and WO97/46527 which were disclosed different crystalline forms and an amorphous form. WO97/46526 claimed the preparation, XRD, and IR, data for four crystalline forms namely, I, II, III, and IV. WO 97/46527 claimed the preparation, XRD, IR, and DSC of crystalline forms I, II, III and IV.

U.S. Pat. No. 6,140,321 discloses the preparation, XRD, IR, TG-DTA, melting points and stability data for crystalline forms I, II, III and IV while for the amorphous form, except the preparation and melting point, other data is disclosed. However, the above patent only claims the preparation, XRD and IR data of Form-III

Further, U.S. Pat. No. 5,985,864 discloses the preparation, XRD, IR, TG-DTA, DSC, melting points and stability data for crystalline forms I, II, III, and IV. The said patent also discloses crystalline form-V whereas no stability or TG-DTA data is disclosed for this crystalline form. However, the above patent, only claimed the preparation, XRD and IR data of polymorphs II, IV and V.

According to example-4 of U.S. Pat. No. 4,895,841, the recrystallization of the crude Donepezil hydrochloride from a mixture of methanol and isopropylether afford the pure donepezil hydrochloride having melting range of 211 to 212° C., no other physicl data was disclosed in the said patent regarding to its crystalline properties, such as XRD, IR, TG-DTA and DSC.

U.S. Pat. No. 6,140,321 and U.S. Pat. No. 5,985,864 discloses, the crystallization of Donepezil hydrochloride using the same solvent combination as said above (i.e., Methanol and isopropylether) afford crystalline form-I having a melting range of 225 to 226° C., and moisture content of 5.17%. Further DSC, TG-DTA data disclosed in the same patent reveals that, form-1 disclosed may be a hydrated crystal form.

Since, polymorphic forms of the drug substances are known to differ in their physical properties such as, melting point, solubility etc., they can appreciably influence the pharmaceutical properties such as dissolution rate and bio availability. However, it is important to further evaluate the polymorphism to obtain new polymorphs exhibiting different dissolution characteristics and in some cases superior bio-availability, stabiity and excellent handling properties.

Also, the stability of this drug against heat and humidity during the storage period is very essential, so a more stable medicinal substance of donepezil hydrochloride is there fore desirable. Hence, the present invention aims to provide a novel crystalline form-VI and the process for its preparation.

### SUMMARY OF THE INVENTION

The present invention is directed to novel crystalline form-VI of Donepezil hydrochloride and the process for preparation thereof which is a simple, non-hazardous and commercially viable process. The novel crystalline form-VI of Donepezil Hydrochloride is specified by the peaks appearing in the powder X-ray diffraction pattern and infrared absorption spectra in potassium bromide.

The present invention for preparing the novel crystalline form-VI of Donepezil hydrochloride comprises the dissolution of the Donepezil free base (which is prepared according to example 3 of our earliear patent application having the reg. No. 555/MAS/02 and which is under process at IPO office India) in a suitable alcoholic solvent and then reacted with HCl source to afford the form-VI of Donepezil hydrochloride.

Optionally the obtained crystalline form-VI of Donepezil hydrochloride can be purified by recrystallization from a mixture of methanol and isopropyl ether.

The novel crystalline form-VI was characterized and analyzed by its powder X-ray diffractogram, infrared absorption spectrum, Differential scanning calorimetery, thermogravimetric analysis, and melting point. Novel crystalline form-VI of Donepezil hydrochloride of the present invention, is easily filterable, anhydrous, stable, high melting, and free flowing crystals.

### BRIEF DESCRIPTION OF THE ACCOMPANYING DRAWINGS

FIG. 1: X-ray powder diffractogram of novel crystalline Form (VI) of Donepezil hydrochloride.

FIG. 2: Infrared spectra of novel crystalline form (VI) of Donepezil hydrochloride.

FIG. 3: Thermogram of TGA of novel crystalline Form (VI) of Donepezil hydrochloride.

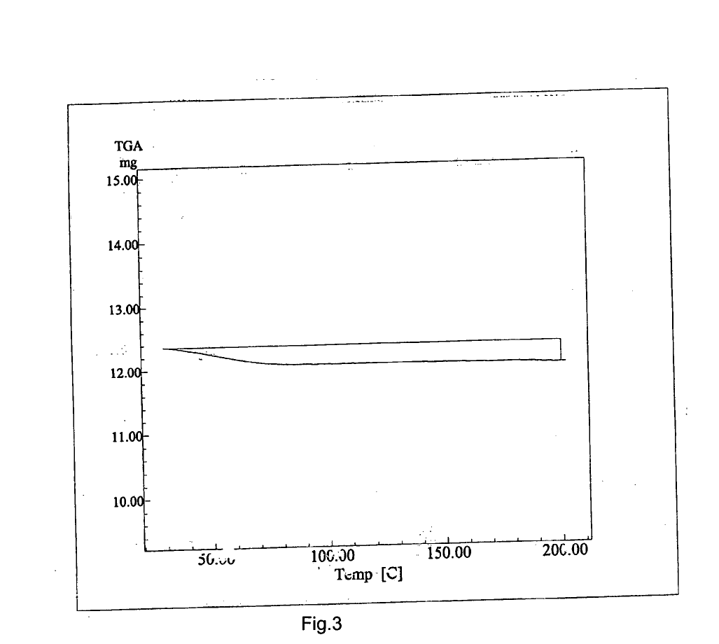

FIG. 4: Differential Scanning Calorimetry thermogram of novel crystalline form(VI) of Donepezil hydrochloride.

### DETAILED DESCRIPTION OF THE INVENTION

The present invention is directed to the novel crystalline form-VI of Donepezil hydrochloride and its preparation thereof, The process for the preparation of novel crystalline form-VI of Donepezil hydrochloride comprises

a. dissolution of the Donepezil free base (which is prepared according to example 3 of our earliear patent application having the reg No. 555/MAS/02 which is under process at IPO office India) in a suitable alcoholic solvent at 60 to 65° C., wherein the said alcoholic solvent may be selected from the group comprising of methanol, ethanol, propanol, and butanol, preferably the said solvent is methanol;

b. reacting the solution of step (a) with HCl source at 25 to 35° C. to afford the Donepezil hydrochloride of crystalline form-VI, where the HCl may be HCl gas purged in ethereal solvents such as isopropyl ether HCl, ethylether HCl, methy tertiary butyl ether HCl, preferably the HCl source may be HCl gas dissolved in isopropyl ether, more preferably stoichiometric amount of HCl gas dissolved in isoprpylether;

c. diluting the reaction mass of step (b) with ethereal solvent, such as diethyl ether, methyl tert-butyl ether, diisopropyl ether; preferably diisopropyl ether;

d. stirring the reaction mass of step (c) at 25 to 35° C. for a period of 0.5 to 10 hours preferably for 2 to 3 hours;

e. filtration of the separated solid from step (d) by conventional methods;

f. drying the resulted crystalline solid from step (e) at 50-55° C. for a period of 5-8 hours under reduced pressure to afford the novel crystalline form-VI of Donepezil hydrochloride;

The novel crystalline form-VI of the present invention is free flowing, non-hydrated and non solvated and hence may be useful in the preparation of pharmaceutical formulations. The novel crystalline form-VI of Donepezil hydrochloride is characterized by XRD, which shows well resolved peaks and the diffractogram is substantially depicted as in FIG. (1). The characteristic peaks (in 2-theta values) and their relative intensities (in %) are given in the following Table-2.

TABLE 2
Crystalline form-VI

| Diffraction angles | Intensity (%) |
|---|---|
| (2θ.°) | (I/Io) |
| 9.742 | 12.1 |
| 11.528 | 9.4 |
| 12.737 | 100.0 |
| 14.220 | 12.8 |
| 14.402 | 22.8 |
| 14.645 | 13.9 |
| 16.176 | 8.7 |
| 16.649 | 21.0 |
| 18.168 | 2.7 |
| 19.303 | 22.1 |
| 20.543 | 20.1 |
| 21.032 | 45.6 |
| 21.491 | 4.0 |
| 22.653 | 59.1 |
| 23.128 | 24.6 |
| 23.837 | 5.1 |
| 24.138 | 5.0 |
| 24.791 | 26.6 |
| 25.152 | 6.4 |
| 25.969 | 4.8 |
| 26.748 | 11.7 |
| 27.272 | 7.3 |
| 27.569 | 5.7 |
| 28.782 | 6.7 |
| 29.937 | 2.7 |
| 30.762 | 3.1 |
| 31.358 | 1.1 |
| 31.956 | 2.1 |
| 32.667 | 3.4 |
| 33.803 | 2.4 |
| 36.272 | 2.1 |

Infrared absorption spectra of Donepezil hydrochloride in potassium bromide having the bands around at: 558.78, 588.84, 607.87, 649.42, 706.31, 749.77, 764.95, 783.81, 810.93, 861.44, 897.21, 927.67, 950.37, 982.24, 1035.44, 1073.41, 1102.41, 1120.94, 1223.44, 1266.49, 1313.99, 1367.83, 1424, 1456.23, 1501.51, 1589.30, 1697.55, 2512.14, 2847.03, 2932.79, 3450.67 cm⁻¹.The IR spectrum is substantially depicted as in FIG. (2).

Melting point of the novel crystalline form VI of Donepezil hydrochloride of the present invention is 222-225° C. (decomposition) which is different from that of the prior art forms. Furthermore the thermogravimetric analysis (TGA) of the crystalline form VI of Donepezil hydrochloride of the present invention measured under the following condition show different patterns from the prior art disclosed crystalline forms. It is noted accordingly that, the novel crystalline form VI is completely different from other crystalline forms disclosed in prior art.

Method and Condition of the Thermogravimetric Analysis (TGA):

About 5-8 mg of samples were taken and subjected to thermal analysis under 5° C./minute of scan speed.

The TGA thermogram of novel crystalline form VI of Donepezil hydrochloride is substantially depicted as in FIG. (3). The novel crystalline form VI of Donepezil hydrochloride has also been characterized by DSC, which exhibits a significant endo peak around 229.85° C. The Differential Scanning Calorimetry thermogram of novel crystalline form-VI of Donepezil hydrochloride is substantially depicted as in FIG. (4).

### EXPERIMENTAL SECTION

Preparation of Novel Crystalline Form VI of Donepezil hydrochloride:

Donepezil free base (10 grams), was dissolved in methanol (50 ml) at a temperature of 60-65° C. along with stirring till a clear solution was obtained. The reaction mass was allowed to cool to a temperature of 25-35° C. along with stirring. Isopropyl ether containing 7.5% of dissolved HCl (20.4 ml, correspond to 1.1 equivalent) was added to the reaction mass at 25 to 35° C. for 10-15 min. Isopropyl ether (80 ml) was added further to reaction mass and stirred for 2.0 hours. The obtained crystalline solid material was filtered, washed with Isopropyl ether (30.0 ml) and dried at a temperature of 50-55° C. under reduced pressure to afford a novel crystalline form VI of Donepezil hydrochloride (9.0 gms).

We claim:

1. Novel crystalline form VI of 1-benzyl-4-[(5,6-dimethoxy-1-indanon)-2-yl]methyl piperidine hydrochloride (Donepezil hydrochloride).

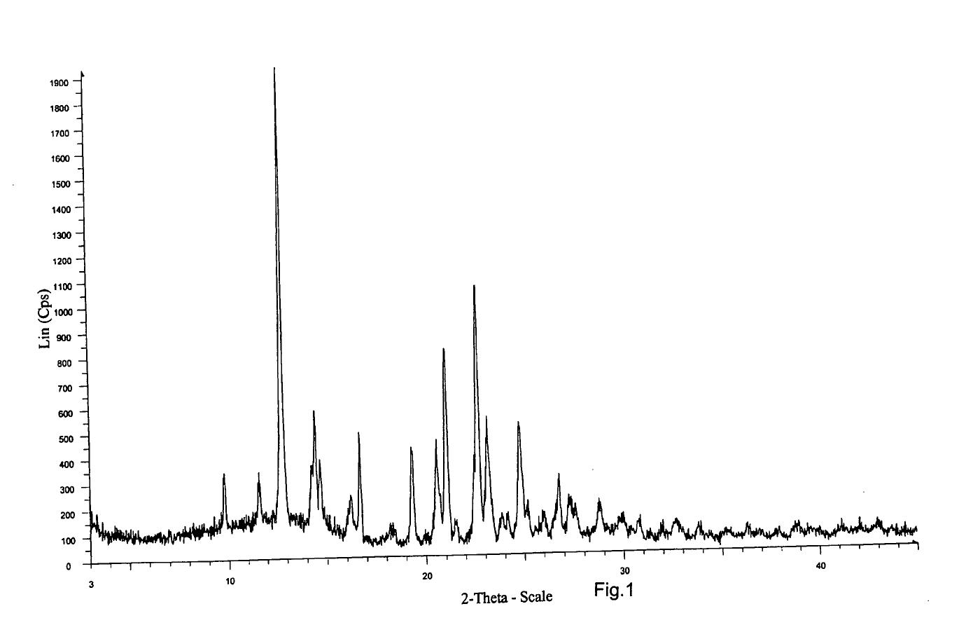

2. The novel crystalline form VI of Donepezil hydrochloride of claim 1 having X-ray powder diffraction pattern with peaks (according to FIG. 1) around 9.742, 11.528, 12.737, 14.220, 14.402, 14.645, 16.176, 16.649, 18.168, 19.303, 20.543, 21.032, 21.491, 22.653, 23.128, 23.837, 24.138, 24.791, 25.152, 25.969, 26.748, 27.272, 27.569, 28.782, 29.937, 30.762, 31.358, 31.956, 32.667, 33.803, 36.272 two-theta degrees.

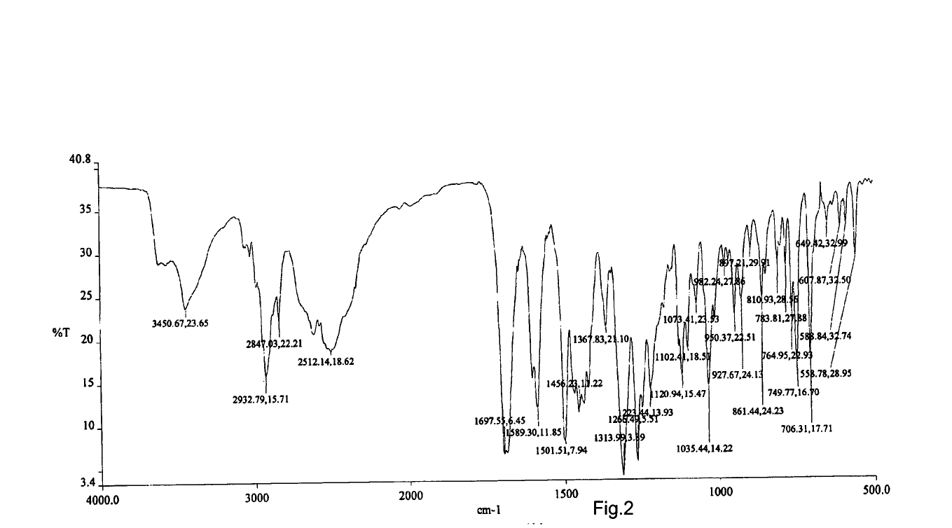

3. The novel crystalline form VI of Donepezil hydrochloride according to claim 1 having an identified characteristic Infrared bands (according to FIG. 2) around 558.78, 588.84, 607.87, 649.42, 706.31, 749.77, 764.95, 783.81, 810.93, 861.44, 897.21, 927.67, 950.37, 982.24, 1035.44, 1073.41, 1102.41, 1120.94, 1223.44, 1266.49, 1313.99, 1367.83, 1424, 1456.23, 1501.51, 1589.30, 1697.55, 2512.14, 2847.03, 2932.79, 3450.67 cm⁻¹.

4. The novel crystalline form VI of Donepezil hydrochloride according to claim 1 having a thermogravimetric analysis thermogram substantially in accordance with FIG. (3).

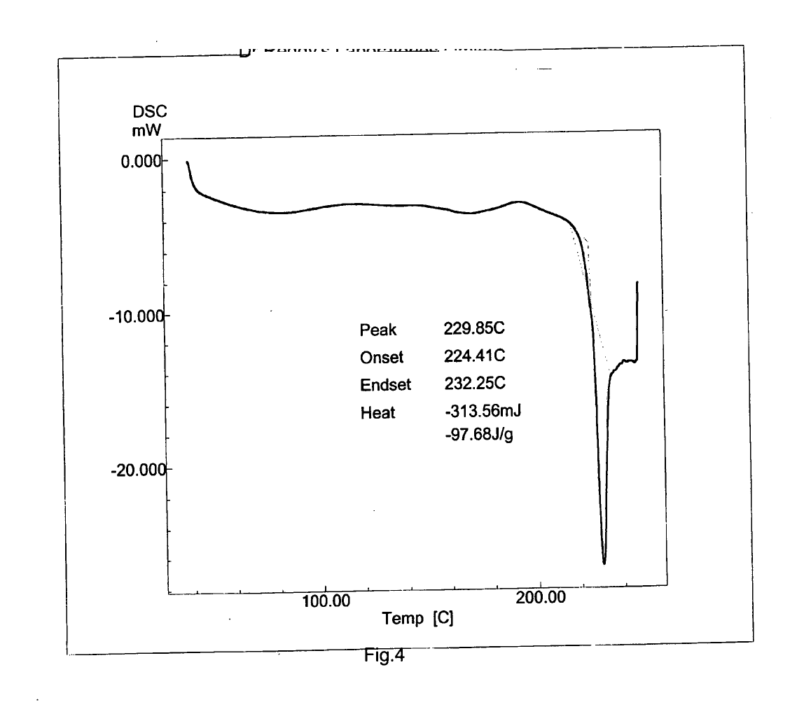

5. The novel crystalline form VI of Donepezil hydrochloride according to claim 1 having a Differential Scanning Calorimetry thermogram (according to FIG. 4) which exhibits a significant endo peak around 229.85° C.

6. A process for preparing the novel, crystalline form (VI) of Donepezil hydrochloride, which comprises;
a. dissolution of the Donepezil free base (which is prepared according to example 3 of our earlier patent application having the reg No. 555/MAS/02 which is under process at IPO office India) in a suitable alcoholic solvent at 60 to 65° C., wherein the said alcoholic solvent may be selected from the group comprising of methanol, ethanol, propanol, and butanol, preferably the said solvent is methanol;
b. reacting the solution of step (a) with HCl source at 25 to 35° C. to afford the Donepezil hydrochloride of crystalline form-VI, where the HCl may be HCl gas purged in ethereal solvents such as isopropyl ether HCl, ethylether HCl, methy tertiary butyl ether HCl, preferably the HCl source may be HCl gas dissolved in isopropyl ether, more preferably stoichiometric amount of HCl gas dissolved in isoprpylether;
c. diluting the reaction mass of step (b) with ethereal solvent, such as diethyl ether, methyl tert-butyl ether, diisopropyl ether; preferably diisopropyl ether;
d. stirring the reaction mass of step (c) at 25 to 35° C. for a period of 0.5 to 10 hours preferably for 2 to 3 hours;
e. filtration of the separated solid from step (d) by conventional methods;
f. drying the resulted crystalline solid from step (e) at 50-55° C. for a period of 5-8 hours under reduced pressure to afford the novel crystalline form-VI of Donepezil hydrochloride;

7. Novel crystalline form VI of 1-benzyl-4-[(5,6-dimethoxy-1-indanon)-2-yl]methylpiperidine hydrochloride (Donepezil hydrochloride) and process for the preparation thereof which is herein described and exemplified.
